(19) United States
(12) Patent Application Publication
(10) Pub. No.: US 2020/0390686 A1
(43) Pub. Date: Dec. 17, 2020

(54) NATURAL SKIN CARE COMPOSITIONS AND METHODS FOR TREATING OXIDATIVE STRESS AND RESTORING SKIN HEALTH

(71) Applicant: CODEX BEAUTY CORPORATION, Portola Valley, CA (US)

(72) Inventors: Marc CORNELL, Cork (IE); Barbara A. PALDUS, Portola Valley, CA (US)

(21) Appl. No.: 16/901,875

(22) Filed: Jun. 15, 2020

Related U.S. Application Data
(60) Provisional application No. 62/861,739, filed on Jun. 14, 2019.

(51) Int. Cl. A61K 8/9794; A61Q 19/08; A61Q 17/04

(52) U.S. Cl. CPC A61K 8/9794; A61Q 19/08; A61K 2800/59; A61K 2800/522; A61Q 17/04

(57) ABSTRACT

The present disclosure is directed to a composition intended for application onto human skin suffering from oxidative stress, the composition comprising: (1) a mixture of at least: (a) a leaf extract of Aristotelia chilensis; (b) a leaf extract of Buddleja globosa; (c) a leaf extract of Ugni molinae; (d) optionally, a bark/seed extract of Entada phaseoloides; (e) optionally, a blend of Pfaffia paniculata, Ptychopetalum olacoides, and Lilium candidum extracts; and (f) at least one humectant; (2) an emulsifier; and (3) a dermatologically acceptable carrier, wherein (a)-(f) are employed in amounts sufficient to synergistically neutralize existing free radicals present in the skin, while proactively priming the skin in order to enhance its ability to protect itself against free radical aggression, thereby helping to enhance its health and appearance, and wherein the composition is natural and free of a skin sensitizing-effective amount of an essential oil.

### FIELD OF THE INVENTION

The present disclosure generally relates to compositions and methods for treating skin suffering from oxidative stress. More particularly, the disclosure is directed to the use of specific associations of botanical extracts and preservative systems to arrive at compositions and methods of enhancing the antioxidant defense potential, hydration, and barrier effect of skin suffering from oxidative stress.

### BACKGROUND

Skin is subject to damage by a number of extrinsic (environmental), and intrinsic factors. Examples of extrinsic factors include exposure to ultraviolet (UV) rays emanating from the sun, as well as harmful chemical agents found in airborne pollution such as smog and cigarette smoke. Intrinsic factors that negatively impact skin include, for example, chronological aging, a person's genetic makeup, and other biological changes that occur from within the skin. These factors cause skin to experience deleterious cellular effects associated with oxidative stress caused by harmful free radicals in the skin. One of the most common free radicals is the reactive oxygen species (ROS) that, in essence, is unstable oxygen molecules.

Many individuals purposefully expose their skin to harmful UV radiation by sunbathing or using tanning beds in an effort to obtain a suntan, considered by many to be a sign of beauty and affluence. Unfortunately, although the immediate effects of ultraviolet radiation may be considered aesthetically and socially gratifying, the long-term hazards including the risk of oxidative stress are cumulative and potentially quite serious, as is evidenced by the size of the global sunscreen market. This market has grown considerably in recent years, with many new products being introduced each and every year. What used to be considered a seasonal business is now viewed as one requiring year-round attention. Sun protection actives, meant to absorb and/or reflect harmful UV rays, are now included in a wide variety of personal products, particularly cosmetic products meant to be worn daily.

Exposure to UV light is known to induce free radical formation in skin. The primary short-term hazard of prolonged exposure to sunlight is erythema, i.e., sunburn. UV rays with wavelengths in the 290 to 320 nanometer range, designated as the UVB wavelength range, tend to be the primary cause of erythema. While the UVA range (320-400 nm) is also known to cause erythema, UV rays of this wavelength are known to play a significant role in the premature aging of skin, a phenomenon colloquially known as photoaging, due to their ability to penetrate more deeply into the skin and cause free-radical formation on a deeper level.

As explained herein, free radicals steal energy from healthy cells in the skin, causing oxidative stress. This in turn activates enzymes in the skin that break down collagen and damage the DNA of a cell, resulting in sunburn and premature aging. Photoaging is characterized by wrinkling and yellowing of the skin, along with other physical changes such as cracking, telangiectasia (spider vessels), solar keratosis (growths), ecchymoses (subcutaneous hemorrhagic lesions), and loss of elasticity (sagging).

Regarding other environmental aging factors such as air pollution like smog and cigarette smoke, chemicals present therein are either themselves free radicals such as, for example, nitrogen dioxide, or have the ability to drive free-radical formation. These free radicals, when present within a biological setting such as the skin, cause a flow of electrons from one molecule to another. The importance of this process lies in the reactivity of the molecules involved.

Under normal conditions, electrons orbit around atoms in pairs, having opposite spins. When an atom has a single unpaired electron, its reactivity increases markedly, at which point it is referred to as a free radical. In a biological setting, free radicals are potentially very dangerous because they can react indiscriminately with neighboring molecules such as proteins, DNA, and vital cell structures such as the cell membrane. This process of electron stealing leads to oxidation. If these reactions are numerous, they can cause extensive cellular damage. The extent of damage depends on the availability of neutralizing antioxidant cellular defenses, as these specialized molecules preferentially react with free radicals, thus neutralizing them.

These cellular defense mechanisms help to reduce the amount of damage that free radicals and reactive species of radicals may cause to the skin by scavenging free radicals or enzymatically converting the free radicals to less toxic chemical species, thereby serving a physiological role, similar to antioxidants. The body's antioxidant defense system can become impaired, however, by the aging process and/or compromised by, for example, inflammation/erythema, infection, and other disorders characterized by oxidative stress.

Oxidative stress has also been found to negatively impact water homeostasis of the skin, i.e., the ability of the skin to maintain constant hydration levels. It is important to the health and appearance of skin to keep it nourished in order to help counteract the damage caused by oxidative stress. Dry skin is a particularly common disorder that affects both males and females equally and is particularly prevalent in older individuals and those genetically predisposed to such a condition. People suffering from dry skin complain of flaking, itching, irritation, and an overall dull, rough, and lackluster appearance to their skin.

Moreover, as a person ages, their skin tends to produce fewer natural oils that aid in preventing moisture from escaping from, and thus dehydrating, the skin. Disruption of water homeostasis occurs at an early stage as a person's skin ages. This is because the expression of aquaporin-3, one of the proteins that regulates water flow at a cellular level, decreases as a person ages. Skin lacking in hydration cannot rid itself of toxins, resulting in irritation and inflammation. Thus, by maintaining good hydration of the surface layers of the skin, epidermis, and horny layer in particular, it becomes possible to substantially enhance the effectiveness of endogenous antioxidant molecules as discussed below, as well as cosmetic agents exhibiting antioxidant properties, in order to help alleviate oxidative stress. In particular, properly hydrated skin slows the aging process by helping to maintain skin's elasticity.

The skin itself has a strong antioxidant defense mechanism to prevent and protect against UV-induced oxidative stress. This is accomplished via endogenous antioxidants such as superoxide dismutase (SOD), catalase, and other molecules like glutathione (GSH) that reduce the formation of ROS prior to the occurrence of oxidative changes in the tissue. The combination of SOD and catalase completely scavenges superoxide ion-initiating ROS. Unfortunately, prolonged UV exposure prevents cutaneous antioxidants such as SOD, catalase, and others from continuously performing these functions, thereby inevitably resulting in oxidative damage to the tissue and unmitigated ROS formation. Eventually, serious skin disorders such as photo-aging and skin cancer can be realized.

Another important defense against oxidative stress is skin's barrier function. The term “barrier function” refers to the outermost layer of the skin, the stratum corneum, which is responsible for keeping moisture in, and keeping damaging elements like UV rays and free radicals out. When the skin's barrier function is operating properly, skin is firm, plump, and hydrated. However, when the barrier function deteriorates, skin health deteriorates as well.

The stratum corneum is the primary line of defense between an individual and the outside world, preventing environmental chemicals and biological irritants from penetrating the skin. For example, free radicals, bacteria, other microbes, allergens, toxic chemicals, UV light, and the like are blocked by the stratum corneum from penetrating into the skin.

While protection against external assaults is a very important function served by the stratum corneum, an even more important function is to prevent the escape of water. The stratum corneum is made up of multiple stacks of flattened cells or “corneocytes,” each of which is encased in a thick coating of fat. If one were to compare the stratum corneum to a brick wall, the stack of cells are bricks, and the fatty matrix encasing them is the mortar. Together, they form a barrier that keeps skin's water content inside so that the skin stays firm, hydrated, elastic, and less prone to wrinkling.

Despite its importance, the barrier formed by the stratum corneum is quite delicate and prone to thinning as a person ages. Moreover, any assault on the stratum corneum, either from external assaults or cellular water loss, can lead to sensitized, dehydrated skin that is susceptible to environmental harm, dryness, irritation, breakout, sagging, and other signs of aging. When skin is dry, it is more permeable to irritants and allergens that can trigger inflammation that in turn can cause rosacea, acne, eczema, and premature aging.

In view of the above, it is clear that in order to both effectively treat skin already suffering from oxidative stress and to protect against further oxidative stress, at least three issues must be addressed. First, any free radicals currently residing in the skin need to be neutralized in order to terminate any existing oxidative stress the skin is experiencing. Second, the skin needs to be rehydrated in order to replace any water/moisture lost as a result of the oxidative stress caused by free radicals present in the skin prior to their being neutralized. Lastly, the skin's barrier function needs to be repaired in order to help protect the skin against further attack by free radicals stemming from both extrinsic and intrinsic factors.

Accordingly, there is a need for skin care compositions and methods that treat or address oxidative stress and restore skin health by neutralizing free radicals in the skin, rehydrating the skin, and repairing the skin's barrier function.

There is no shortage of conventional cosmetic products in the market meant to enhance the health and appearance of skin by combatting the negative effects associated with the influence of both extrinsic and intrinsic factors. However, the cosmetic industry has recently embraced a sub-category of these products deemed to be organic/natural, and there is a current trend by consumers towards these types of goods. These products are believed to possess health and environmental benefits. In line with the philosophy of such products, consumers also expect them to be paraben-free, phthalate-free, sulfate-free, silicone-free, synthetic fragrance-free, alcohol-free, phenoxyethanol-free, or otherwise non-toxic. This category of organic/natural products has become one of the fastest growing in the global personal care and cosmetic segments.

In response to the outstanding need in the industry for products that meet certain thresholds of “natural” and “organic” ingredients, coupled with the lack of official standards for what qualifies as “natural” and “organic,” preservative formulation has become a cottage industry with consumers gravitating towards products containing natural extracts, botanicals, or other ingredients derived from natural sources, while avoiding those products having ingredients that are either known to cause or suspected of causing adverse health reactions. Unfortunately, this ad hoc approach and decentralization of acquired knowledge and experience of generating effective preservative formulations has led to a host of ineffective solutions that typically result in diminished shelf-life and usability of associated cosmetic consumer products.

Various third-party certifications have been established in an attempt to bring consistency and reliability to the use of natural and organic preservatives in topical consumer products. For example, ECOCERT® is an organic certification organization based in Europe that conducts inspections in over 80 countries, making it one of the largest organic certification organizations in the world. ECOCERT® primarily certifies food and food products but also certifies cosmetics, detergents, perfumes, and textiles, and is a leading certifier of fair-trade food, cosmetics, and textiles.

Another example is the Cosmetic Organic Standard (COSMOS), a Europe-wide private standard that was developed by five charter members: BDIH (Germany), Cosmebio (France), Ecocert Greenlife SAS (France), ICEA (Italy), and Soil Association (Great Britain). They were all combined under an AISBL (international non-profit organization based in Brussels), the purpose of which was to set out minimum common requirements, harmonize organic and natural cosmetic certification rules, and lobby institutions in the sector's interests. COSMOS makes use of the principles in the ECOCERT® standard: to promote the use of ingredients from organic farming, use production and manufacturing processes that are environmentally sound and safe for human health, and include and expand the concept of “green chemicals.”

The National Organic Program (NOP), a federal regulatory framework in the United States governing organic food, is yet another certification. The core mission of the NOP is to protect the integrity of the United States Department of Agriculture (USDA) organic seal. The seal is used for products adhering to USDA standards that contain at least 95% organic ingredients.

Hence, the industry has increased its efforts to develop “natural” cosmetic formulations using non-synthetic ingredients. This approach differs from the synthetic ingredient-based approach that has allowed the cosmetic industry to develop cosmetics with consistent product integrity, performance, and shelf life through the use of harsh, irritating, synthetic ingredients such as phenoxyethanol.

Accordingly, there is a need for skin care compositions and methods for treating or addressing oxidative stress and restoring skin health that are natural and free of harsh, irritating, synthetic ingredients while providing effective broad-spectrum preservative protection as well as promoting or cooperating with ingredients for treating oxidative stress.

The use of botanical extracts on skin, in general, is known. However, based on only the sheer number of botanical extract candidates in existence, together with extraction techniques and solvents that may be used, a virtually infinitesimal number of products can be formulated with no assurance that the composition made will be both stable and useful for its desired purpose. Consequently, the ability to formulate skin treatment products, in the absence of skin-sensitizing ingredients, that are natural, highly efficacious and stable, is a daunting challenge as the inventors have discovered. One cannot merely combine a mixture of random botanical extracts, in arbitrary concentrations, using arbitrary extraction techniques and solvents, with the expectation that all of the disparate ingredients contained therein will be both compatible to one another, and yield the intended benefits and properties.

For example, U.S. Pat. No. 4,933,177 discloses the use of certain botanical ingredients for application onto skin. However, the reference is devoid of any specific teaching or suggestion regarding the precise association of ingredients, extraction techniques and solvents to be used, as well as which types of ingredients are to be avoided, in order to formulate an efficacious, natural product capable of enhancing skin health and appearance.

Similarly, U.S. Pat. No. 7,678,768; GB 2485483; WO 2006/032091; WO 2012/033422; WO 2013/149323; and WO 2019/002714 all disclose botanical ingredients for application onto skin for a plethora of potential uses. However, not only do these references disclose a small sample size of puzzle-piece candidates available to a formulator, but when one also considers the amounts in which each of these ingredients may be used, together with all the other variables that must be taken into consideration when formulating with plant extracts, to say that successfully arriving at a targeted product is like finding the proverbial needle in a haystack, is indeed an understatement.

One of the major deterrents associated with the use of botanical ingredients in skin care compositions relates to their relative instability in products as evidenced by loss of potency, odor deviations, and discoloration. These negative attributes increase the risk of microbiological contamination and proliferation, instability, and inadequate safety of the products. This problem becomes even more acute when the composition has to qualify as being “natural.” The elimination of conventionally used synthetic, inorganic, and/or petroleum-derived ingredients from a formulator's toolbox severely hampers their ability to make efficacious, yet stable, skin treatment products. One might argue that a formulator skilled in the art could determine, through routine experimentation, which botanical extracts, auxiliary ingredients, excipients, solvents, and amounts of each can be combined in order to arrive at an intended product. However, as was mentioned above, in view of the sheer number of combinatorial permutations that exist, based on the number of ingredients that may be chosen, causes the successful formulation of such a product to be based more on luck and happenstance, as opposed to routine experimentation.

Based on the foregoing, it is an object of embodiments of the present disclosure to provide natural, organic and ECOCERT®-approved skin care compositions and methods that are effective at treating and priming skin suffering from oxidative stress.

Another object of embodiments of the present disclosure is to provide natural, organic and ECOCERT®-approved compositions and methods capable of proactively priming the skin and enhancing its ability to defend itself against free-radical aggression.

Another object of embodiments of the present disclosure is to provide skin care compositions and methods providing effective broad-spectrum anti-microbial activity using natural ingredients while treating or addressing oxidative stress and/or priming the skin against free-radical aggression.

### SUMMARY

The present disclosure is directed to a composition intended for application onto human skin, the composition that includes (1) a mixture of at least: (a) a leaf extract of Aristotelia chilensis; (b) a leaf extract of Buddleja globosa; (c) a leaf extract of Ugni molinae; and (d) optionally, a bark/seed extract of Entada phaseoloides; (e) optionally, a blend of Pfaffia paniculata, Ptychopetalum olacoides, and Lilium candidum extracts; and (f) at least one humectant; (2) an emulsifier; and (3) a dermatologically acceptable carrier, wherein (a)-(f) are employed in amounts sufficient to synergistically neutralize existing free radicals present in the skin, while proactively priming the skin in order to enhance its ability to defend itself against free-radical aggression, thereby improving its health and appearance, and wherein the composition is natural.

According to another embodiment, the present disclosure is also directed to a composition intended for application onto human skin, the composition that includes (1) a preservative system; (2) a mixture of at least: (a) a leaf extract of Aristotelia chilensis; (b) a leaf extract of Buddleja globosa; and (c) a leaf extract of Ugni molinae; and (d) optionally, a bark/seed extract of Entada phaseoloides; (e) optionally, a blend of Pfaffia paniculata, Ptychopetalum olacoides, and Lilium candidum extracts; and (f) at least one humectant; (3) an emulsifier; and (4) a dermatologically acceptable carrier, wherein (a)-(f) are employed in amounts sufficient to synergistically neutralize existing free radicals present in the skin, while proactively priming the skin in order to enhance its ability to defend itself against free-radical aggression, thereby improving its health and appearance, and wherein the composition is natural, free of a skin-sensitizing amount of an essential oil, and has a pH ranging from about 4.5 to about 5.5, and preferably from about 4.8 to about 5.3.

The present disclosure is also directed to a method of treating and priming skin suffering from or at risk of suffering from oxidative stress in order to enhance its health and appearance by applying one of the above-disclosed compositions onto the skin.

According to another embodiment, the present disclosure is also directed to a method of proactively priming the skin and enhancing its ability to defend itself against future free-radical aggression.

According to yet another embodiment, the present disclosure is directed to a natural preservative system that cooperates with a skin care composition and method for treating or addressing oxidative stress. The preservative system may be a preservative system as discussed in co-pending application Ser. No. 16/669,045, filed Oct. 30, 2019, and incorporated herein in its entirety by reference.

According to yet another embodiment, the present disclosure is directed to a skin care composition intended for application to the skin as a sun block or sunscreen product.

These and other features, aspects and advantages of the present disclosure will become evident to those skilled in the art from a reading of the present disclosure.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates levels of superoxide dismutase activity in response to different compositions including components of embodiments of the disclosure.

FIG. 2 illustrates levels of superoxide dismutase activity in response to different compositions including components of embodiments of the disclosure.

FIG. 3 illustrates transmissions spectra of components of embodiments of the disclosure.

FIG. 4 illustrates the results of a microbial challenge assay using an exemplary all-natural preservative system in a composition according to an embodiment of the disclosure.

FIG. 5 illustrates the results of a microbial challenge assay using an exemplary all-natural preservative system in a composition according to another embodiment of the disclosure.

FIG. 6 illustrates the results of a microbial challenge assay using an exemplary all-natural preservative system in a composition according to another embodiment of the disclosure.

### DETAILED DESCRIPTION

For purposes of the present disclosure, the use of the word “natural” is intended to encompass ECOCERT®-approved ingredients or formulations synonymous with the terms “green,” “clean,” “organic,” “sustainable,” “eco-friendly,” or “environmentally-friendly” as known and used in the art. The term “natural,” for example, may be used in the context of holistic or homeopathic formulations and is intended to include those topical consumer products and/or preservative systems that are plant-based, paraben-free, and/or non-toxic.

Further, when used in the context of the antimicrobial properties of a preservative or preservative system, the term “broad spectrum” is intended to describe those preservatives or preservative systems of the present disclosure that have the ability to inhibit the growth of or kill a wide range of microorganisms that decay or spoil topical consumer products. For example, a “broad spectrum” preservative system inhibits the growth of or kills a wide range of bacteria and fungi, preferably a wide range of Gram-positive and Gram-negative bacteria, yeasts, molds, and/or other fungi.

The compositions of the present disclosure can comprise, consist essentially of, or consist of, the components of the present disclosure as well as other ingredients described herein. The term “comprising” as used herein is meant to include various optional, compatible components that can be used in the preservative systems and cosmetic compositions of the present disclosure without limiting the inclusion, use of, or cooperation with other ingredients, excipients, uses, or otherwise. The term “consisting essentially of” as used herein means that the composition or component may include additional ingredients, but only if the additional ingredients do not materially alter the basic and novel characteristics of the compositions or methods.

As used herein, the words “preferred,” “preferably,” and variants thereof refer to embodiments of the disclosure that afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the disclosure.

Numerical ranges as used herein are intended to include every number and subset of numbers contained within that range, whether specifically disclosed or not. Further, these numerical ranges should be construed as providing support for a claim directed to any number or subset of numbers within that range.

All percentages, parts, proportions, and ratios as used herein are by weight of the total composition, unless otherwise specified. All such weights as they pertain to listed ingredients are based on the active level.

All references to singular characteristics or limitations of the present disclosure shall include the corresponding plural characteristic or limitation, and vice versa, unless otherwise specified or clearly implied to the contrary by the context in which the reference is made.

All publications, articles, papers, patents, patent publications, and other references cited herein are hereby incorporated in their entireties for all purposes to the extent consistent with the disclosure herein.

The terms “prime” and “priming” as described herein refer to the process of rehydrating/moisturizing and repairing the barrier function of human skin in order to proactively protect it from future oxidative stress.

The term “oxidative stress” as described herein refers to the disturbance in balance between reactive oxygen species (ROS) and/or free radicals and antioxidants present in the skin caused by extrinsic and/or intrinsic factors. Extrinsic factors include, for example, exposure to UV radiation, pollution, and products containing harsh chemicals. Intrinsic factors include, for example, chronological aging, a person's genetic makeup, and other biological changes that occur from within the skin.

The term “skin-sensitizing effective amount” as described herein is meant to exclude an amount of a volatile essential oil that can lead to an allergic response following contact with an individual's skin. Skin sensitization is an immunological response to previous exposure to a substance that results in an inflammatory skin reaction. An allergic skin reaction is usually presented as a red, itchy, bumpy rash. Examples of the types of volatile essential oils that can cause skin sensitization, depending on their amounts within a skin care composition include, but are not limited to, frankincense, myrrh, and sweet orange.

The term “free radicals” as described herein refers to those ROS that are formed when skin experiences oxidative stress caused by extrinsic factors including exposure to UV radiation and environmental stressors such as pollution and harmful chemical agents typically found, for example, in hard-surface cleaning products.

The present disclosure generally relates to compositions and methods for effectively alleviating oxidative stress in order to enhance human skin's health and appearance, while at the same time or alternatively enabling the skin to defend itself against further free-radical aggression. Moreover, the composition is also natural, organic, and ECOCERT®-approved, thus being free of synthetic and/or petroleum-derived ingredients.

It has surprisingly been discovered by the inventors that a composition that is both natural and free of a skin sensitizing-effective amount of an essential oil, comprising a mixture of specific botanical infusions comprising at least: (a) a leaf extract of Aristotelia chilensis; (b) a leaf extract of Buddleja globosa; (c) a leaf extract of Ugni molinae; (d) optionally, a bark/seed extract of Entada phaseoloides; and (e) optionally, a blend of Pfaffia paniculata, Ptychopetalum olacoides, and Lilium candidum extracts, when applied onto skin, synergistically alleviates oxidative stress in order to perfect the skin's health and appearance, while at the same time priming the skin to enable it to protect itself against future free radical aggression.

Aristotelia chilensis leaf extract is derived from the leaves of a small dioecious evergreen tree in the Elaeocarpaceae family native to South America in the Valdivian temperate rainforests of Chile, which also goes by the name maqui. The extract has been found to contain high amounts of anthocyanins, indole alkaloids, and flavonoids. These compounds serve as a source of antioxidants that help to neutralize free radicals and protect the skin's DNA.

This extract has also been found to be rich in the anthocyanidin delphinidin. In a study entitled “NADPH oxidase is a novel target of delphinidin for the inhibition of UVB-induced MMP-1 expression in human dermal fibroblasts,” Lim T G, Jung S K, Kim Y, Lee H J, Jang T S, Lee K W, John Wiley & Sons Ltd, Experimental Dermatology, 2013, 22, 417-437, it was reported that delphinidin was effective at inhibiting UVB-induced MMP-1 expression in the skin, which is known to cause degradation of dermal collagen. Various enzyme systems in the skin are associated with endogenous ROS production, including the enzyme system NADPH oxidase (NOX), which plays a key role in triggering ROS production. Studies have shown that NOX activation is thus closely related to ROS-induced skin aging. The study concluded that delphinidin significantly inhibits UVB-induced MMP-1 expression in human dermal fibroblasts, which then inhibits NOX enzyme activation, which in turn inhibits ROS production, and therefore this particular anthocyanidin might prevent photoaging.

This extract is commercially available from N-Active EIRL under the trade name EthniCare® MAQUI.

The Aristotelia chilensis leaf extract is preferably employed in an amount of from about 1 to about 10% by weight, and most preferably from about 2 to about 5% by weight, based on the total weight of the composition.

Buddleja globosa leaf extract is derived from the leaves of the orange ball buddleja, a.k.a. matico, a species of flowering plant endemic to Chile and Argentina. The extract has been found to contain glycosidic flavonoids and phenylethanoids such as verbascoside, iridoids, triterpenoids, and di- and sesquiterpenoids, together with two caffeic acid derivatives. These compounds have shown promise in wound healing due to their ability to promote fibroblast growth, with a strong antioxidant effect. This particular leaf extract is also rich in stigmasterol, an unsaturated plant sterol found in plant oils.

In an article entitled “Analgesic, anti-inflammatory, and antioxidant properties of Buddleja globosa, Buddlejaceae,” Backhouse N, Rosales L, Apablaza C, Goïty L, Erazo S, Negrete R, Theodoluz C, Rodriguez J, Delporte C J, Ethnopharmacol. 2008 Mar. 5; 116(2):263-9, it was reported that plant extracts having fractions rich in stigmasterol and β-sitosterol display anti-inflammatory properties. This extract is commercially available from N-Active EIRL, under the trade name EthniCare® MATICO.

The Buddleja globosa leaf extract is preferably employed in an amount of from about 0.5 to about 3% by weight, and most preferably from about 1 to about 2% by weight, based on the total weight of the composition.

Ugni molinae leaf extract is derived from the leaves of a woody evergreen shrub from the myrtaceae family commonly found in Chile, and is also known by its Spanish name murta. The extract has been found to contain various phenolic compounds including gallic acid, catechin, quercetin, myricetin, and kaempferol. These compounds have been found to possess strong antioxidant activity against ROS production, lipid peroxidation, and superoxide anion production.

In an article entitled “MURTA (Ugni molinae Turcz.): A REVIEW ON CHEMICAL COMPOSITION, FUNCTIONAL COMPONENTS AND BIOLOGICAL ACTIVITIES OF LEAVES AND FRUITS,” Lopez J, Vega-Galvez A, Rodriguez A, Uribe E, Bilbao-Sainz C, Chilean J. Agric. Anim. Sci., ex Agro-Ciencia (2018) 340:1-14, the Ugni molinae leaf extract was found to have large amounts of flavonoids (anthocyanins, flavonols, flavanols), condensed and hydrolysable tannins, stilbenoids (resveratrol), and phenolic acids, which possess both antioxidant and antimicrobial activity. As was mentioned previously, anthocyanins have been found to possess antioxidant/free radical scavenging properties that prevent oxidative stress, a phenomenon shown to negatively affect skin health and appearance.

The Ugni molinae leaf extract is preferably employed in an amount of from about 0.5 to about 3% by weight, and most preferably from about 1 to about 2% by weight, based on the total weight of the composition. This extract is commercially available from N-Active EIRL, under the trade name EthniCare® MURTA.

Entada phaseoloides bark/seed extract is derived from the seeds of a woody, evergreen vine from the Fabaceae family found in Africa, Asia, Australia, and the western Pacific. Its primary molecules include Entadamide A and Phaseoloidin. Entadamide A limits urocanic acid isomerization in skin, thereby inhibiting inflammation and immunosuppression, while also serving as a UV absorber. Phaseoloidin, a homogentisic acid glucoside, is a molecule with superior free-radical scavenging ability.

In a study entitled, “Experimental evaluation of anti-inflammatory effect of topical application of entada phaseoloides seeds as paste and ointment,” Dawane J, Pandit V, Rajopadhye B, N Am J Med Sci. 2011 November; 3(11): 513-517, the topical application of a paste and ointment containing the Entada phaseoloides bark/seed extract was confirmed as having potent anti-inflammatory properties.

The Entada phaseoloides bark/seed extract may be employed in an amount of from about 1 to about 5% by weight, and preferably in an amount of from about 2 to about 3% by weight, based on the total weight of the composition. This extract is commercially available from Biosil Technologies, Inc. headquartered in Allendale, N.J., under the trade name Entadine®.

The blend of the present disclosure comprises Pfaffia paniculata, Ptychopetalum olacoides, and Lilium candidum extracts. Pfaffia paniculata root extract is derived from a plant in the Amaranthaceae family typically found in South America. It is an extract characterized by the presence of vitamins, minerals, amino acids, phytosterols, pfaffic acid, pfaffosides, allantoin, mucilage, and saponins. This extract has been found to possess anti-inflammatory, immuno-stimulant, and analgesic properties.

Ptychopetalum olacoides bark/stem extract is derived from a flowering plant in the Olacaceae family indigenous to central Amazonian forests. The extract, which possesses antioxidant properties, is characterized by the presence of alkaloids, resinous materials rich in organic acids and tannins, traces of essential oils, sterols, triterpenic alcohols, and lupeol.

Lilium candidum extract is derived from the bulbs and flowers of the Liliaceae family. It is characterized by the presence of amino acids, flavonoids, glycosides, and steroids, and has been found to possess antifungal and anti-inflammatory properties. The blend of Pfaffia paniculata, Pytchopetalum olacoides, and Lilium candidum extracts is commercially available from Chemyunion LTDA, a Brazilian company headquartered in Sao Paulo, under the name Bioskinup™ Contour 3R.

The blend may be employed in embodiments of the disclosure in an amount of from about 1 to about 5% by weight, and preferably in an amount of from about 2 to about 3% by weight, based on the total weight of the composition.

The composition of the present disclosure further includes at least one humectant in order to further enhance the hydration and moisturization of the skin, thereby providing enhanced priming. The humectant will typically be employed in an amount of from about 1.0 to about 6.0% by weight, and preferably from about 1.5 to about 4.0% by weight, based on the total weight of the composition. Examples of suitable humectants include, but are not limited to, hyaluronic acid and its derivatives such as sodium hyaluronate and hydrolyzed hyaluronic acid, lecithin, aloe vera, panthenol, glycerin, and seaweed. A particularly preferred humectant for use in embodiments of the skin care compositions and methods of the present disclosure is hydrolyzed hyaluronic acid.

According to one embodiment of the present disclosure, there is provided a composition intended for application onto human skin suffering or at risk of suffering from oxidative stress, the composition comprising: (1) a mixture of at least: (a) from about 1 to about 10% by weight, and preferably from about 2% to about 5% by weight, of a leaf extract of Aristotelia chilensis; (b) from about 0.5 to about 3% by weight, and preferably from about 1 to about 2% by weight, of a leaf extract of Buddleja globosa; (c) from about 0.5 to about 3% by weight, and preferably from about 1 to about 2% by weight, of a leaf extract of Ugni molinae; (d) optionally, from about 1 to about 5% by weight, and preferably from about 2 to about 3% by weight, of a bark/seed extract of Entada phaseoloides; (e) optionally, from about 1 to about 5% by weight, and preferably from about 2 to about 3% by weight, of a blend of Pfaffia paniculata, Ptychopetalum olacoides, and Lilium candidum extracts; and (f) from about 1 to about 8% by weight, and preferably from about 2 to about 6% by weight, of at least one humectant, all weights based on the total weight of the composition; (2) an emulsifier; and (3) a dermatologically acceptable carrier, wherein (a)-(f) are employed in amounts sufficient to neutralize existing free radicals present in the skin, as well as defend against further free-radical aggression, thereby enhancing the skin's health and appearance, and wherein the composition is natural.

In another embodiment of the present disclosure, the inventors have surprisingly discovered that a natural preservative system comprising a combination of specific amounts of: a Lactobacillus ferment, a Lactobacillus and Cocos mucifera (coconut) fruit extract, salicylic acid (in some embodiments optional), a salt of a weak acid such as potassium sorbate, and propanediol which is optionally petroleum-free, when incorporated into a composition having a specific pH range, effectively both prohibits and inhibits microbial growth on and in the composition.

The Lactobacillus ferment of the present disclosure is preferably employed in an amount of from about 1 to about 5% by weight, preferably from about 2 to about 4% and more preferably from about 2 to about 4%, by weight of the total composition. A “Lactobacillus ferment” may refer to the solution obtained after fermentation of a defined growth medium by the bacterium Lactobacillus spp. During fermentation, Lactobacillus bacteria produce antimicrobial peptides that can provide broad spectrum antimicrobial protection at appropriate concentrations and/or in combination with other antimicrobial agents. An exemplary Lactobacillus ferment is commercially available from Active Micro Technologies under the tradename Leucidal® SF.

The Lactobacillus and Cocos mucifera fruit extract can include any Cocos mucifera fruit extract fermented with Lactobacillus and/or included with Lactobacillus ferment of the present disclosure and is preferably employed in an amount of from about 1 to about 5%, preferably from about 2 to about 4%, by weight of the total composition. “Cocos mucifera fruit extract fermented with Lactobacillus,” may reference the solution obtained after Lactobacillus fermentation of Cocos mucifera (coconut) fruit extract instead of a defined growth medium. The result is a materially different antimicrobial product that is effective at preventing the growth of fungi, specifically yeasts and molds, at appropriate concentrations and/or in combination with other antimicrobial agents. An exemplary Lactobacillus and Cocos mucifera extract is commercially available from Active Micro Technologies under the tradename Amticide® Coconut and is typically associated with the International Nomenclature of Cosmetic Ingredients (INCI) name of a Lactobacillus and Cocos mucifera (coconut) fruit extract.

When present, salicylic acid is preferably employed in an amount of up to about 0.5% by weight, preferably from about 0.1 to about 0.45% and more preferably from about 0.2 to about 0.4%, by weight of the total composition. It should be noted that the use of salicylic acid in an amount at or greater than about 0.5% by weight, based on the total weight of the composition, renders the composition a drug requiring FDA approval prior to commercialization and sale in the United States. In some embodiments, salicylic acid may be omitted by adjusting the concentrations of Lactobacillus ferment, Lactobacillus and Cocos mucifera fruit extract, and/or other ingredients as described in greater detail herein.

The salt of a weak acid is preferably employed in an amount of up to about 0.5% by weight, preferably from about 0.1 to about 0.45% and more preferably from about 0.2% to about 0.4%, by weight of the total composition. A preferred salt of a weak acid is potassium sorbate (i.e., the potassium salt of sorbic acid). Other weak acids that may be used in their salt form include, but are not limited to, acetic acid, propionic acid, and benzoic acid.

Propanediol, such as a petroleum-free 1,3-propanediol, is typically employed in an amount of about 1% to about 10% by weight, preferably from about 2% to about 8% and more preferably from about 4% to about 6%, by weight of the total composition. An exemplary petroleum-free 1,3-propanediol is commercially available from Dupont Tate & Lyle Bio Products under the tradename Zemea® Propanediol and can be associated with the INCI name propanediol.

The inventors have unexpectedly discovered that the ability of the preservative system of the present disclosure to effectively inhibit microorganism growth is critically dependent on the pH of the composition in which it is used. For example, if the preservative system is employed in a composition having a pH of 6, it fails to provide the requisite broad-spectrum protection needed for acceptable storage stability/shelf-life. Accordingly, the pH of a composition comprising the preservative system of the present disclosure may be in a range of from about 4.5 to about 5.5, and preferably from about 4.8 to about 5.3.

According to this embodiment of the present disclosure, there is provided a composition intended for application onto human skin in order to enhance its health and appearance, the composition comprising: (1) a preservative system that includes: (a) from about 1 to about 5%, preferably from about 2 to about 4% by weight, of a Lactobacillus ferment; (b) from about 1 to about 5%, preferably from about 2 to about 4% by weight, of a Lactobacillus and Cocos mucifera fruit extract; (c) up to about 0.5% by weight, preferably from about 0.1 to about 0.45, and most preferably from about 0.25 to about 0.4% by weight of salicylic acid; (d) from about 0.1 to about 0.5% by weight, and preferably from about 0.2 to about 0.4% by weight of at least one salt of a weak acid, preferably potassium sorbate; and (e) from about 1 to about 10%, preferably from about 2 to about 8% by weight, and most preferably from about 4 to about 6% by weight of 1,3-propanediol; (2) a mixture of at least: (f) from about 1 to about 10% by weight, and preferably from about 2 to about 5% by weight, of a leaf extract of Aristotelia chilensis; (g) from about 0.5 to about 3% by weight, and preferably from about 1 to about 2% by weight, of a leaf extract of Buddleja globosa; (h) from about 0.5 to about 3% by weight, and preferably from about 1 to about 2% by weight, of a leaf extract of Ugni molinae; (i) optionally, from about 1 to about 5% by weight, and preferably from about 2 to about 3% by weight, of a bark/seed extract of Entada phaseoloides; (j) optionally, from about 1 to about 5% by weight, and preferably from about 2 to about 3% by weight, of a blend of Pfaffia paniculata, Ptychopetalum olacoides, and Lilium candidum extracts; and (k) from about 1 to about 8% by weight, and preferably from about 2 to about 6% by weight, of at least one humectant, all weights based on the total weight of the composition; (3) an emulsifier; and (4) a dermatologically acceptable carrier, wherein (f)-(k) are employed in amounts sufficient to neutralize existing free radicals present in the skin, and/or to defend against further free-radical aggression, thereby enhancing the skin's health and/or appearance, and wherein the composition is natural, free of a skin-sensitizing amount of an essential oil, and has a pH ranging from about 4.5 to about 5.5, and preferably from about 4.8 to about 5.3.

In another embodiment of the present disclosure, there is provided a composition intended for application onto human skin in order to enhance its health and appearance, the composition comprising: (1) a preservative system that includes: (a) from about 1 to about 5%, preferably from about 2 to about 4% by weight, of a Lactobacillus ferment; (b) from about 1 to about 5%, preferably from about 2 to about 4% by weight of a Cocos mucifera fruit extract fermented with Lactobacillus; (c) from about 0.1 to about 0.45% by weight, preferably from about 0.25 to about 0.4% of salicylic acid; (d) up to about 0.5% by weight, preferably from about 0.2 to about 0.4% by weight, of at least one salt of a weak acid, preferably potassium sorbate; and (e) from about 1 to about 10%, preferably from about 2 to about 8% by weight, and most preferably from about 4 to about 6% by weight, of a petroleum-free propanediol; (2) a mixture of at least: (f) from about 1 to about 10% by weight, and preferably from about 2 to about 5% by weight, of a leaf extract of Aristotelia chilensis; (g) from about 0.5 to about 3% by weight, and preferably from about 1 to about 2% by weight, of a leaf extract of Buddleja globosa; (h) from about 0.5 to about 3% by weight, and preferably from about 1 to about 2% by weight, of a leaf extract of Ugni molinae; (i) optionally, from about 1 to about 5% by weight, and preferably from about 2 to about 3% by weight, of a bark/seed extract of Entada phaseoloides; (j) optionally, from about 1 to about 5% by weight, and preferably from about 2 to about 3% by weight, of a blend of Pfaffia paniculata, Ptychopetalum olacoides, and Lilium candidum extracts; and (k) from about 1 to about 8% by weight, and preferably from about 2 to about 6% by weight, of at least one humectant, all weights based on the total weight of the composition; (3) an emulsifier; and (4) a dermatologically acceptable carrier, wherein (f)-(k) are employed in amounts sufficient to neutralize existing free radicals present in the skin, and/or to defend against further free-radical aggression, thereby enhancing the skin's health and/or appearance, and wherein the composition is natural, free of a skin-sensitizing amount of an essential oil, and has a pH ranging from about 4.5 to about 5.5, and preferably from about 4.8 to about 5.3.

According to yet another embodiment of the present disclosure, there is provided a composition intended for application onto human skin in order to enhance its health and appearance, the composition comprising: (1) a preservative system that includes: (a) from about 1 to about 5%, preferably from about 2 to about 4% by weight, of a Lactobacillus ferment; (b) from about 1 to about 5%, preferably from about 2 to about 4% by weight, of a Cocos mucifera fruit extract fermented with Lactobacillus; (c) from about 0.1 to about 0.45%, preferably from about 0.25 to about 0.4% by weight, of salicylic acid; (d) from about 0.1 to about 0.5% by weight, preferably from about 0.2 to about 0.4% by weight, of at least one salt of a weak acid, preferably potassium sorbate; and (e) from about 1 to about 10%, preferably from about 2 to about 8%, and most preferably from about 4 to about 6% by weight, of a petroleum-free 1,3-propanediol; (2) a mixture of at least: (f) from about 1 to about 10% by weight, and preferably from about 2 to about 5% by weight, of a leaf extract of Aristotelia chilensis; (g) from about 0.5 to about 3% by weight, and preferably from about 1 to about 2% by weight, of a leaf extract of Buddleja globosa; (h) from about 0.5 to about 3% by weight, and preferably from about 1 to about 2% by weight, of a leaf extract of Ugni molinae; (i) optionally, from about 1 to about 5% by weight, and preferably from about 2 to about 3% by weight, of a bark/seed extract of Entada phaseoloides; (j) optionally, from about 1 to about 5% by weight, and preferably from about 2 to about 3% by weight, of a blend of Pfaffia paniculata, Ptychopetalum olacoides, and Lilium candidum extracts; and (k) from about 1 to about 8% by weight, and preferably from about 2 to about 6% by weight, of at least one humectant, all weights based on the total weight of the composition; (3) an emulsifier; and (4) a dermatologically acceptable carrier, wherein (f)-(k) are employed in amounts sufficient to neutralize existing free radicals present in the skin, and/or to defend against further free-radical aggression, thereby enhancing the skin's health and/or appearance, and wherein the composition is natural, free of a skin-sensitizing amount of an essential oil, and has a pH ranging from about 4.5 to about 5.5, and preferably from about 4.8 to about 5.3.

In yet another embodiment of the present disclosure, there is provided a composition intended for application onto human skin in order to enhance its health and appearance, the composition comprising: (1) a preservative system comprised of: (a) from about 2 to about 4% by weight, of a Lactobacillus ferment; (b) from about 2 to about 4% by weight, of a Cocos mucifera fruit extract fermented with Lactobacillus; (c) from about 0.25 to about 0.4% by weight, of salicylic acid; (d) from about 0.2 to about 0.4% by weight, of at least one salt of a weak acid, preferably potassium sorbate; and (e) from about 4 to about 6% by weight, of a petroleum-free 1,3-propanediol; (2) a mixture of at least: (f) from about 1 to about 10% by weight, and preferably from about 2 to about 5% by weight, of a leaf extract of Aristotelia chilensis; (g) from about 0.5 to about 3% by weight, and preferably from about 1 to about 2% by weight, of a leaf extract of Buddleja globosa; (h) from about 0.5 to about 3% by weight, and preferably from about 1 to about 2% by weight, of a leaf extract of Ugni molinae; (i) optionally, from about 1 to about 5% by weight, and preferably from about 2 to about 3% by weight, of a bark/seed extract of Entada phaseoloides; (j) optionally, from about 1 to about 5% by weight, and preferably from about 2 to about 3% by weight, of a blend of Pfaffia paniculata, Pytchopetalum olacoides, and Lilium candidum extracts; and (k) from about 1 to about 8% by weight, and preferably from about 2 to about 6% by weight, of at least one humectant, all weights based on the total weight of the composition; (3) an emulsifier; and (4) a dermatologically acceptable carrier, wherein (f)-(k) are employed in amounts sufficient to neutralize existing free radicals present in the skin, and/or to defend against further free-radical aggression, thereby enhancing the skin's health and/or appearance, and wherein the composition is natural, free of a skin-sensitizing amount of an essential oil, and has a pH ranging from about 4.5 to about 5.5, and preferably from about 4.8 to about 5.3.

According to another embodiment of the present disclosure, there is provided a composition intended for application onto human skin in order to enhance its health and appearance, the composition comprising: (1) a preservative system comprised of: (a) from about 2 to about 4% by weight, of a Lactobacillus ferment; (b) from about 2 to about 4% by weight, of a Cocos mucifera fruit extract fermented with Lactobacillus; (c) from about 0.25 to about 0.4% by weight, of salicylic acid; (d) from about 0.2 to about 0.4% by weight, of at least one salt of a weak acid, preferably potassium sorbate; and (e) from about 4 to about 6% by weight, of a petroleum-free 1,3-propanediol; (2) a mixture of at least: (f) from about 1 to about 10% by weight, and preferably from about 2 to about 5% by weight, of a leaf extract of Aristotelia chilensis; (g) from about 0.5 to about 3% by weight, and preferably from about 1 to about 2% by weight, of a leaf extract of Buddleja globosa; (h) from about 0.5 to about 3% by weight, and preferably from about 1 to about 2% by weight, of a leaf extract of Ugni molinae; (i) optionally, from about 1 to about 5% by weight, and preferably from about 2 to about 3% by weight, of a bark/seed extract of Entada phaseoloides; (j) optionally, from about 1 to about 5% by weight, and preferably from about 2 to about 3% by weight, of a blend of Pfaffia paniculata, Pytchopetalum olacoides, and Lilium candidum extracts; and (k) from about 1 to about 8% by weight, and preferably from about 2 to about 6% by weight, of at least one humectant, all weights based on the total weight of the composition; (3) an emulsifier; and (4) a dermatologically acceptable carrier, wherein (f)-(k) are employed in amounts sufficient to neutralize existing free radicals present in the skin, and/or to defend against further free-radical aggression, thereby enhancing the skin's health and/or appearance, and wherein the composition is natural, free of a skin-sensitizing amount of an essential oil, and has a pH ranging from about 4.5 to about 5.5, and preferably from about 4.8 to about 5.3.

According to another embodiment of the present disclosure, there is provided a composition intended for application onto human skin in order to enhance its health and appearance, the composition comprising: (1) a preservative system comprised of: (a) about 4% by weight, of a Lactobacillus ferment; (b) about 4% by weight, of a Cocos mucifera fruit extract fermented with Lactobacillus; (c) about 0.4% by weight, of potassium sorbate; and (e) about 4% by weight, of a petroleum-free 1,3-propanediol; (2) a mixture of at least: (f) from about 1 to about 10% by weight, and preferably from about 2 to about 5% by weight, of a leaf extract of Aristotelia chilensis; (g) from about 0.5 to about 3% by weight, and preferably from about 1 to about 2% by weight, of a leaf extract of Buddleja globosa; (h) from about 0.5 to about 3% by weight, and preferably from about 1 to about 2% by weight, of a leaf extract of Ugni molinae; (i) optionally, from about 1 to about 5% by weight, and preferably from about 2 to about 3% by weight, of a bark/seed extract of Entada phaseoloides; (j) optionally, from about 1 to about 5% by weight, and preferably from about 2 to about 3% by weight, of a blend of Pfaffia paniculata, Ptychopetalum olacoides, and Lilium candidum extracts; (k) from about 1 to about 8% by weight, and preferably from about 2 to about 6% by weight, of at least one humectant, all weights based on the total weight of the composition; (3) an emulsifier; and (4) a dermatologically acceptable carrier, wherein (f)-(j) are employed in amounts sufficient to neutralize existing free radicals present in the skin, and/or to defend against further free-radical aggression, thereby enhancing the skin's health and/or appearance, and wherein the composition is natural, free of a skin-sensitizing amount of an essential oil, and has a pH ranging from about 4.5 to about 5.5, and preferably from about 4.8 to about 5.3.

The dermatologically acceptable carrier can encompass a wide variety of forms. In some cases, the solubility or dispersibility of the components in the composition may dictate the form and character of the carrier. Non-limiting examples include simple solutions (e.g., aqueous or anhydrous), dispersions, emulsions, and solid forms. In certain embodiments, the dermatologically acceptable carrier is in the form of an emulsion. An emulsion can be generally classified as having a continuous aqueous phase (e.g., oil-in-water and water-in-oil-in-water) or a continuous oil phase (e.g., water-in-oil or oil-in-water). While the oil phase may comprise any vegetable oil, so long as it does not cause skin sensitization, a particularly preferred oil component is almond oil.

The inventors have surprisingly discovered that the use of almond oil enables compounds present in the botanical extract to effectively penetrate into the skin, without having to use skin-sensitizing essential oils, while still facilitating the desired degree of efficacy. This is due to almond oil being rich in beta-zoosterol, squalene, and alpha-tocopherol, together with lesser amounts of carbohydrates, proteins, vitamins, and minerals such as vitamin B complex (comprising vitamins B1, B2, B3, B5, B6, B7, B9, B12) and zinc. Moreover, almond oil's phytochemicals are believed to be effective at promoting surface level proliferation and skin cell development. Other oils that may also be used include, but are not limited to, vegetable oils such as olive oil, jojoba oil, babassu oil, castor oil, coconut oil, corn oil, cotton seed oil, linseed oil, mustard oil, safflower oil, sesame oil, soybean oil, sunflower seed oil, wheat germ oil, argan oil and manila oil.

Any ingredient capable of emulsifying the composition may be employed as an emulsifier without departing from the spirit of the disclosure, so long as it is natural and/or dermatologically acceptable. Examples thereof include, but are not limited to, glyceryl stearate, cetyl alcohol, sodium stearoyl lactylate, sorbitan olivate, cetearyl olivate, cetearyl alcohol, cetearyl glucoside, sodium cetearyl sulfate, and the like. It is also particularly preferred that the emulsifier be free of palm oil.

The compositions of the present disclosure may be made available to consumers in a wide variety of product forms that include, but are not limited to, solutions, suspensions, lotions, creams, gels, sprays, ointments, foams, and serums. For example, a product intended for application onto skin, post-shaving, in order to help relieve the irritation associated with the mechanical stress on the skin caused by the shaving process, can be formulated using the above-described compositions as a base formula.

In an embodiment, a product intended for application onto skin prior to or during exposure to sunlight, in order to serve as a sunscreen or sun block, may be formulated according to the above-described compositions as a base formula.

According to embodiments of the present disclosure, the compositions can also additionally comprise suitable optional ingredients as desired. For example, the composition can optionally include other active or inactive ingredients, provided they do not unacceptably alter the benefits of the skin care composition, are natural, and/or do not promote skin sensitization. The precise amount of optional ingredients will be determined by those skilled in the art.

Examples of optional additive ingredients that may be employed include, but are not limited to, humectants, emollients, flavonoids, minerals, chelating agents, pH regulators/buffers, rheology modifiers, phytosterols, vitamin B3 compound, anti-inflammatory agents such as licorice extracts, bisabolol, manjistha extracted from plants in the genus Rubia, guggal extracted from plants in the genus Commiphora, Quillaja saponaria extract, kola extract, chamomile, red clover extract, sea whip extract, hibiscus extract, lucuma extract, ficus extract, red algae extract, sea kale extract, Iceland Moss extract, Saskatoon Berry extract, Siberian Ginseng extract, spruce needles extract, birch bark extract, yarrow extract, marigold extract, and couch grass extract.

Additional ingredients that may be employed in order to further potentiate the disclosure's efficacy may include, for example, Peumus boldus (Boldo) leaf extract, Astrocaryum murumuru seed butter, Butyrospermum parkii (shea) butter, Theobroma grandiflorum seed butter, Spondias mombin pulp extract, Mangifera indica pulp extract, Musa sapientum pulp extract, Mauritia flexuosa fruit oil, Physalis angulata extract, Xylityl sesquicaprylate, Vaccinium myrtillus seed oil, Cucubita pepo seed extract, linoleic acid, linolenic acid, Centella asiatica leaf extract, Tamarindus indica seed polysaccharide, Zanthoxylum bungeanum fruit extract, Lactococcus ferment lysate, Bellis perennis flower extract, Coffea arabica seed cake extract, Coffea arabica seed oil, cotton seed oil, linseed oil, Pichia ferment lysate filtrate, and whey protein.

A particularly preferred optional ingredient for use in the composition of the present disclosure is an emollient which may be employed in an amount of from about 1 to about 15% by weight, preferably from about 2 to about 5% by weight, and all weights therebetween. Examples of preferred emollients include, but are not limited to, seed butters such as Astrocaryum murumuru seed butter and Theobroma grandiflorum seed butter. These seed butters provide an enhanced degree of emollience to the compositions of the present disclosure. An especially preferred seed butter is Astrocaryum murumuru seed butter.

In addition to the above-mentioned ingredients, certain types of auxiliary ingredients may also be added to the composition of the present disclosure in order to prophylactically inhibit free-radical formation caused by UV radiation, which induces oxidative stress in the skin, in order to facilitate photoinhibition.

Examples of such auxiliary ingredients include, but are not limited to, pongamia glabra (karanj a) seed oil derived from the pongolote tree, Dunaliella salina algae extract which is rich in beta-carotene, Haematococcus pluvialis algae extract which is rich in astaxanthin, red algae which is rich in mycosporine-like amino acids, zinc oxide, and titanium dioxide.

The Peumus boldus (Boldo) leaf extract, when employed, will typically be used in an amount of from about 0.1 to about 5.0% by weight, such as from about 0.5 to about 3.0% by weight, and from about 1.0 to about 2.0% by weight, based on the weight of the composition.

In yet another embodiment of the present disclosure, there is provided a method of treating and/or priming skin suffering from oxidative stress or at risk of suffering from oxidative stress in order to enhance its health and/or appearance, by applying one of the above-disclosed compositions onto the skin.

A further embodiment of the present disclosure provides for a method of proactively priming and enhancing human skin's ability to defend itself against future free radical aggression by applying one of the above-disclosed compositions onto the skin.

### EXAMPLES

The following examples as set forth herein are intended for illustrative purposes only and are not intended to limit the scope of the disclosure in any way, as many variations thereof are possible without departing from the spirit and scope of the disclosure. In the examples, all concentrations are listed as weight percent, unless otherwise specified.

### Example 1

A mixture of: Aristotelia chilensis+Buddleja globosa+Ugni molinae leaf extracts (hereinafter, “EthniCare® M3”) was evaluated for total glutathione quantification and superoxide dismutase (SOD) activity, in a lysate cell via an enzymatic assay. The absorbance reading was performed at 410 nm for total glutathione and 450 nm for SOD using a Multiscan GO monochromator available from Thermo Fisher Scientific of Waltham, Mass. The quantification values were normalized by the total protein in the sample using the Bradford technique, as described in Anal. Biochem, 72:248-254.

Primary human fibroblasts were seeded in 75 cm² flasks, cultured, and expanded in an incubator at 37° C. in the presence of 5% CO2. Upon reaching confluency, the cells were seeded in well plates and exposed to hydrogen peroxide and the quantification mediators.

For statistical evaluation, an ANOVA test was used to initially measure the variation in the results, after which a Bonferroni post-test was used to make the ANOVA results even more precise. A 5% significance level was used.

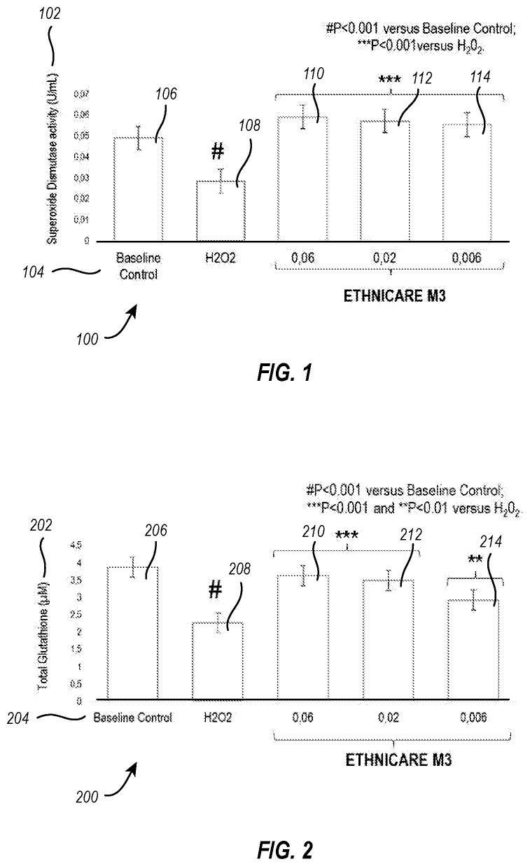

The bar graph 100 of FIG. 1 shows the amount of SOD activity in human fibroblasts exposed to varying amounts of EthniCare® M3 110, 112, 114, as compared to the oxidative stress group H₂O₂ 108 and a baseline control 106 (P<0.001).

Here, it is seen that EthniCare® M3 110, 112, 114 resulted in a significant increase in protective effect against oxidative stress, at various concentration levels, based on increased SOD activity, with a standard deviation of 3. Hence, whereas oxidative stress caused by exposing the human fibroblasts to H₂O₂ 108 resulted in a decrease of SOD activity, subsequent exposure by EthniCare® M3 increased SOD activity by approximately 100%.

With regards to total glutathione production in human fibroblasts, the bar graph 200 in FIG. 2 below further corroborates the protective effect obtained when using EthniCare® M3.

Here too it is seen that EthniCare® M3 results in a significant increase in human fibroblast protective effect against oxidative stress, at various concentration levels 210, 212, 214 of EthniCare® M3, based on increased total glutathione production, with the same standard deviation of 3. Hence, whereas oxidative stress to human fibroblasts caused by H₂O₂ 208 exposure resulted in a decrease of total glutathione, subsequent exposure to EthniCare® M3 210, 212, 214 significantly increased total glutathione production.

The results in FIGS. 1 and 2 demonstrate the significant antioxidant activity realized by EthniCare® M3, in view of its ability to strengthen cellular antioxidant capacity by modulating the antioxidant mediators SOD and total glutathione.

### Example 2

In this example, the photo-protective effect of EthniCare® M3 against visible light ranging from 400-700 nm was evaluated, since long-term exposure to visible light has been shown to induce oxidative stress that can contribute to unwanted skin pigmentation (dark spots), inflammation, and oxidative photo-aging. Visible blue radiation (440-485 nm), also referred to as “blue light,” has the ability to penetrate deeper into the skin than both UVA and UVB light, all the way to the dermis where collagen and elastin reside. Aside from the sun, electronic devices such as smart phones and computer screens emit blue light that most people are exposed to, in large quantities, on a daily basis. The potential negative photo-aging consequences such exposure can have on skin is troubling.

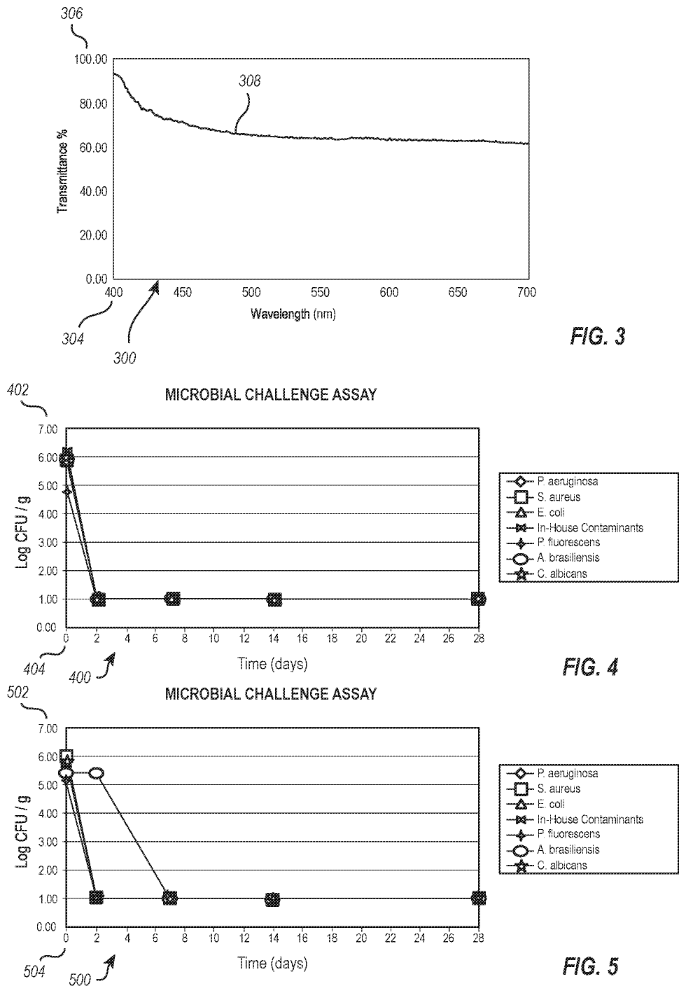

In order to assess its blocking ability, a thin film of EthniCare® M3 was applied onto a Helioplate HD6 substrate from HelioScreen Labs. Transmission spectra were then obtained using a UV 2450 Shimadzu spectrophotometer with an integration sphere (ISR-240A), available from Shimadzu Corporation of Kyoto, Japan. The spectral sweep range was 400-700 nm with measurements taken three times to ensure accuracy, the results of which are found in FIG. 3.

As can be seen from the graph 300 of FIG. 3, the average transmittance 306 of blue light in the range of 440-485 nm, i.e. the UV range, is approximately 67%, with the mean being approximately 69%. Thus, approximately 31%, or one-third, of the blue light radiation was effectively blocked by EthniCare® M3, further evidencing its ability to inhibit oxidative stress and photo-aging damage.

### Example 3

Leaf extracts Aristotelia chilensis and Ugni molinae were evaluated to determine their ability to prevent undesirable thymidine dimer formation upon exposure to UVB radiation. When skin is exposed to UVB radiation, thymidine bases in the DNA can absorb the UVB radiation to form thymidine dimers (also known as thymine dimers) in DNA, complicating the cell's efforts to replicate DNA and to properly function. In this experiment, an in-vitro thymidine dimer assay was performed using a skin model consisting of normal human-derived epidermal keratinocytes cultured to form a multilayered model of the human epidermis.

One group of tissue was treated for seven days with the Aristotelia chilensis leaf extract, another group of tissue was tested for seven days with the Ugni molinae leaf extract, and third group was left untreated. All three groups tissue groups were then exposed to UVB radiation at 300 mJ/cm². DNA was then extracted and assayed for thymidine dimer content.

The DNA was then immobilized and incubated with an antibody specific to thymidine dimers. The primary antibody was then detected with a secondary antibody conjugated to a fluorescent dye. The membrane was then scanned with an excitation laser and emission filter combo specific to the fluorescent dye, so that the fluorescence intensity of each sample was proportional to the amount of thymidine dimers present in the sample and a lower fluorescence intensity advantageously indicates lower levels of cellular damage from UVB radiation. The results of the thymidine dimer assay are found in Table 1, below, expressed as mean Relative Fluorescence Units (RFU)±standard deviation.

TABLE 1

| | Corrected RFU |
|---|---|
| No UVB exposure | 514 ± 57.30 |
| Untreated | 5.463 ± 80.50 |
| 50 ug/ml Trolox (analog of vitamin E) | 4.380 ± 393.00 |
| Aristotelia chilensis at 5% | 3.418 ± 673.10 |
| Ugni molinae at 4% | 4.246 ± 426.30 |

The data in Table 1 shows that tissue treated with Aristotelia chilensis at 5% concentration resulted in a decrease of thymidine dimer formation of approximately 41%, and tissue treated with Ugni molinae at 4% concentration resulted in a decreased formation of thymidine dimer of approximately 25%, as compared to the untreated tissue, thus evidencing their photo-protective effect against UVB-induced DNA damage.

### Example 4

In this example, EthniCare® M3 was evaluated to determine its effect on cutaneous erythema (i.e. sunburn). A total of 10 volunteers were recruited, each of which were subjected to mechanically induced erythema caused by the application and removal of transparent medical tape on their forearm for 20 successive repetitions. Erythema measurements were performed using a Mexameter® MX18 and Multiprobe Adapter MPA-5 available from Courage & Khazaka electronic GmbH, of Koln, Germany. An initial measurement was taken immediately after mechanical insult, followed by two additional measurements at intervals of 30 and 60 minutes. The results showed that all of the volunteers treated with EthniCare® M3 experienced a 41% reduction in erythema after 30 minutes, and a 76% reduction in erythema after 60 minutes, thereby evidencing the ability of EthniCare® M3 to soothe skin suffering from externally caused erythema.

These results show that use of EthniCare® M3 provides a soothing action to skin suffering from erythema.

### Example 5

In this example, EthniCare® M3 was evaluated to determine its effect on the production of nitric oxide in epidermal keratinocytes in culture. It is well known that the skin's exposure to external aggressors such as UV radiation, pollutants, chemical irritants, aesthetic treatments, and the like can often cause inflammatory reactions, resulting in erythema and edema, both of which are painful and unsightly. One of the factors that signals inflammation is nitric oxide (NO) produced endogenously by a variety of cells in order to regulate physiological processes such as neurotransmission, smooth muscle contractility, platelet reactivity, and cytotoxic activity of immune cells. High levels of NO have been found in pathologies such as rheumatoid arthritis and chronic intestinal inflammation, just to name a few.

Cultured epidermal keratinocytes (PAM 212 keratinocytes) were treated at three concentration levels of EthniCare® M3: 1%, 3%, and 5%. The cultured cells were first caused to express nitric oxide synthase leading to the production of NO from L-arginine. The control was a selective inhibitor of NO synthase, added to the cultured cells that completely (100%) inhibited NO production. NO production was measured using a colorimeter. The results obtained showed that: at 1% EthniCare® M3 inhibited 58.0% of NO production; at 3% it inhibited 46.2%; and at 5% it inhibited 25.3%, evidencing its effectiveness at alleviating skin inflammation.

### Example 6

A composition in accordance with the present disclosure was evaluated to determine its ability to successfully pass a micro preservative efficacy testing (PET) challenge. The composition tested is found in Table 2 below.

TABLE 2

| Ingredient | Amount (wt %) |
|---|---|
| EthniCare ® M3 | 1.00 |
| Lactobacillus ferment | 4.00 |
| Lactobacillus and cocos nucifera | 2.00 |
| fruit extract ferment | |
| Propanediol | 4.00 |
| Sodium benzoate | 0.30 |
| Potassium sorbate | 0.20 |
| Citric acid | 1.25 |
| Auxiliaries | 23.13 |
| water | 64.12 |

A graphical representation of the results of a microbial challenge assay performed for the exemplary skin care composition listed in Table 2 above is illustrated in FIG. 4. As seen in the graph 400 of FIG. 4, which shows the log of colony forming units (CFU)/g sample 402 as a function of days 404 after beginning the microbial challenge assay, the composition reduced each of Pseudomonas aeruginosa, Staphylococcus aureus, Escherichia coli, In-House Contaminants, Pseudomonas fluorescens, Aspergillus brasiliensis, and Candida albicans by day 2, with no subsequent uptick in any of the microbial species. The above test results confirm the successful preservation of the composition using the preservative system of the present disclosure in combination with EthniCare® M3.

### Example 7

A composition in accordance with another embodiment the present disclosure was evaluated to determine its ability to successfully pass a micro PET challenge. The composition tested is found in Table 3 below.

TABLE 3

| Ingredient | Amount (wt %) |
|---|---|
| EthniCare ® M3 | 2.00 |
| Blend (Pfaffia paniculata, | 2.00 |
| Ptychopetalum olacoides, | |
| and Lilium candidum extracts) | |
| propanediol | 4.00 |
| Essential oils | 0.44 |
| glycerin | 2.00 |
| Lactobacillus ferment | 4.00 |
| Lactobacillus and cocos nucifera | 2.00 |
| fruit extract ferment | |
| Propanediol | 4.00 |
| Sodium benzoate | 0.30 |
| Potassium sorbate | 0.20 |
| Citric acid | 0.15 |
| Auxiliaries | 11.65 |
| water | 71.26 |

As seen in the graph 500 of FIG. 5, which shows the log CFU/g sample 502 as a function of days 504 after beginning the microbial challenge assay, the composition reduced each of Pseudomonas aeruginosa, Staphylococcus aureus, Escherichia coli, In-House Contaminants, Pseudomonas fluorescens, and Candida albicans by day 2, and Aspergillus brasiliensis by day 7, with no subsequent uptick in any of the microbial species. The above test results confirm the successful preservation of the composition using the preservative system of the present disclosure in combination with EthniCare® M3.

### Example 8

A composition in accordance with the present disclosure was evaluated to determine its ability to successfully pass a micro (PET) challenge. The composition tested is found in Table 4 below.

TABLE 4

| Ingredient | Amount (wt %) |
|---|---|
| EthniCare ® M3 | 2.00 |
| Entadine ® | 2.00 |
| propanediol | 4.00 |
| Essential oils | 0.34 |
| glycerin | 2.00 |
| emollient | 14.00 |
| Lactobacillus ferment | 4.00 |
| Lactobacillus and cocos nucifera | 2.00 |
| fruit extract ferment | |
| Propanediol | 4.00 |
| Sodium benzoate | 0.30 |
| Potassium sorbate | 0.20 |
| Citric acid | 0.15 |
| Auxiliaries | 11.65 |
| water | 71.26 |

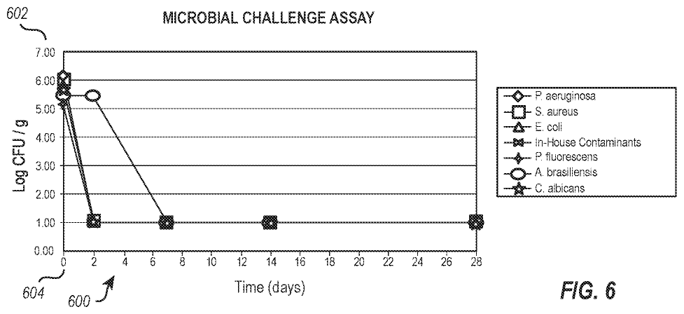

As seen in the graph 600 of FIG. 6, which shows the log CFU/g sample 602 as a function of days 604 after beginning the microbial challenge assay, the composition reduced each of Pseudomonas aeruginosa, Staphylococcus aureus, Escherichia coli, In-House Contaminants, Pseudomonas fluorescens, and Candida albicans by day 2, and Aspergillus brasiliensis by day 7, with no subsequent uptick in any of the microbial species. The above test results confirm the successful preservation of the composition using the preservative system of the present disclosure in combination with Ethnicare® M3, Entadine®, and/or other ingredients of a skin care composition for the treatment of oxidative stress according to the embodiments of the present disclosure.

### Example 9

The ingredients Peumus boldus (Boldo) leaf extract, Buddleja globosa (Matico) leaf extract, Aristotelia chilensis (Maqui) leaf extract, and Ugni molinae (Murta) leaf extract were evaluated to determine what, if any, gene expression effects they may indicate after UVB exposure, per the below-indicated protocol.

Reconstructed Human Epidermis (RHE) tissues were obtained from ZenBio (Research Triangle Park, N.C.; lot #RHE051820) and were used immediately. Tissues were transferred to 6 well plates and were equilibrated for an hour in 1 ml of pre-warmed medium/well ZenSkin provided by the ZenBio. Samples of the above ingredients were then added non-diluted, in triplicates at 3 mg/cm² with the positive displacement pipette and were spread evenly on top of the RHE tissues. Sterile distilled water was the negative control. Tissues were allowed to incubate for a three-hour period with test materials, afterward were transferred to a new 24 well plate, and exposed to 30 mJ/cm² (equivalent to 1 minimal erythema dose, or MED) UVB (302 nm) using a Hoefer (Holliston, Mass.) transilluminator. Tissues were then placed back into the original six well plates with medium and allowed to incubate overnight.

At the end of the incubation RNA was extracted and purified with RNeasy Mini Kit cat.#74104 from Qiagen (Germantown, Md.), using a QiaCube Connect robotic station (Qiagen). Purified total RNA was assessed at 260 nm and 280 nm with a Thermo Fisher Scientific (Waltham, Mass.) NanoDrop™ Lite Spectrophotometer.

cDNA was prepared using a High-Capacity RNA-to-cDNA™ Kit (Applied Biosystems, Thermo Fisher) and the expression of the genes of interest was measured by real-time quantitative PCR with a BioRad iCycler iQ Detection System using PCR primers from Realtimeprimers (Elkins Park, Pa.) and AzuraView GreenFast qPCR Blue Mix LR available from Azura Genomics (Raynham, Mass.). Efficiency ΔΔCt method was used for quantification of results, after the normalization of gene expression to HPRT1 and GAPDH (housekeeping genes).

Genes were considered differentially expressed if the p value, as determined by the two-tailed t-test, was ≤0.10 and the modulation was ≥1.8.

Tissues treated with Boldo exhibited an anti-inflammatory (downregulation of CD44, MAP3K7, PTGS2, MMP1, IL8, IL1A; upregulation of TIMP2), anti-apoptotic (downregulation of FOXO3), anti-hyperproliferative (decrease of MAP3K7, PCNA, END1), vasculoprotective (upregulation of HSPG2, ITGB1), and barrier-protective (upregulation of AQP3, TGM1, LOR, downregulation of CD44) response. The decrease of UVB-induced proinflammatory signal by Boldo evidences a reduction in the need for antioxidant response (downregulation of TXNRD1 and perhaps CAT), with no apparent xenobiotic metabolism response.

Tissue treated with Matico decreased the expression of genes coding for late differentiation proteins (IVL, FLG, CDSN) and possibly increased cell survival (BIRCS), evidencing a potential photo-protective effect.

Tissue treated with Maqui showed Maqui to be a highly bioactive substance. It was found to support barrier-protection and ceramide production by way of its strong upregulation of AQP3 and LOR, as well as FLG and GBA, together with a decrease of HAS3 which is expected in differentiated layers of the epithelium and promoted by ascorbic acid, together with anti-inflammatory properties as evidenced by a significant decrease of IL-4, IL-6, and IL-8 activity.

Maqui was also found to trigger a decrease of the expression of AGER, which is the receptor for AGE (advanced glycation endproducts)—a major contributor to skin aging. Maqui also appeared to exhibit pigmentation-promoting effects through the inhibition of ASIP. Other modulated genes include VEGFA (stimulation of blood vessel growth and therefore skin oxygenation), TIMP (MMP inhibition), and TLR2 (upregulation), whose expression is important for repair of insults, such as those caused by UVB irradiation. Maqui-treated tissues also yielded significant amounts of RNA indicative of a broad photo-protective effect of that test material against UVB-induced cytotoxicity which is supported by the strong downregulation of the pro-apoptotic, UVB-induced FOXO3.

Tissue treated with Murta showed a complex bioactivity profile with pro-inflammatory effects (significant increase of ILIA, PTSG2, EDN1) combined with increased expression of genes coding for proteins important for stratum corneum formation (DSG3, TGM1, LOR) and skin repair after UVB irradiation (TLR2). Moreover, CTGF was significantly upregulated showing its potential for increasing ECM (extracellular matrix) production.

Tissue treated with the combination of Matico, Maqui, and Murta exhibited unusually robust bioactivity. Both the VEGFA and PPARD genes were upregulated, evidencing the biologic signaling initiated via the application of the combination of Matico, Maqui, and Murta. This type of immune system related cellular communication is the skin's biological response to environmental stressors. Environmental stressors related to UV radiation exposure can enable oxidative stress and attendant free radical formation on the skin. Similarly, both the IL8 and MT2A genes were down regulated evidencing the triggering of those biological processes of the skin associated with reducing inflammation caused by UV radiation exposure (i.e., anti-inflammatory effect). Lastly, the SMPD1, TGM1 and AQP3 genes were upregulated indicating that the combination triggered a biological process evidencing an enhancement of the skin's barrier function post-UV radiation exposure (i.e., skin priming function).

The above-referenced examples establish the efficacy of the disclosed embodiments to enhance skin suffering from oxidative stress caused by external stressors such as UV radiation and environment pollution, as well as to prime the skin in order to enhance its barrier function capability, thereby allowing it to better defend itself against external aggressors. The embodiments may advantageously comprise one or a combination of a Peumus boldus (Boldo) leaf extract, Buddleja globosa (Matico) leaf extract, Aristotelia chilensis (Maqui) leaf extract, and Ugni molinae (Murta) leaf extract, which may synergistically enhance, prime, and/or relieve the skin.

By providing a skin care composition and methods according to the present disclosure, the problem of existing skin care compositions and methods intended for treatment of oxidative stress comprising non-natural ingredients, harsh synthetic preservatives, and other harmful components is addressed. The embodiments of the present disclosure advantageously provide a composition effective for neutralizing free radicals in the skin and/or priming the skin for in order to enhance its ability to defend itself against future free-radical aggression.

The skin care composition and method embodiments address the problem of existing skin care treatment modalities being non-natural and/or being inadequate for treating skin conditions such as oxidative stress.

What is claimed is:

1. A composition intended for application onto human skin suffering from oxidative stress, the composition comprising: (1) a mixture of at least: (a) a leaf extract of Aristotelia chilensis; (b) a leaf extract of Buddleja globosa; (c) a leaf extract of Ugni molinae; (d) optionally, a bark/seed extract of Entada phaseoloides; (e) optionally, a blend of Pfaffia paniculata, Ptychopetalum olacoides, and Lilium candidum extracts; and (f) at least one humectant; (2) an emulsifier; and (3) a dermatologically acceptable carrier, wherein (a)-(f) are employed in amounts sufficient to neutralize existing free radicals present in the skin, while proactively priming the skin in order to enhance its ability to defend itself against future free radical aggression, thereby improving its health and appearance, and wherein the composition is natural and free of a skin sensitizing-effective amount of an essential oil.

2. The composition of claim 1, wherein (a) is employed in an amount of from about 1 to about 10% by weight; (b) is employed in an amount of from about 0.5 to about 3% by weight; (c) is employed in an amount of from about 0.5 to about 3% by weight; (d) is employed in an amount of from about 1 to about 5% by weight; (e) is employed in an amount of from about 1 to about 5% by weight; and (f) is employed in an amount of from about 1.0 to about 8.0% by weight, all weights based on the total weight of the composition.

3. The composition of claim 1, wherein (a) is employed in an amount of from about 1 to about 10% by weight; (b) is employed in an amount of from about 0.5 to about 3% by weight; (c) is employed in an amount of from about 0.5 to about 3% by weight; (d) is employed in an amount of from about 1 to about 5% by weight; and (f) is employed in an amount of from about 1.0 to about 6.0% by weight, all weights based on the total weight of the composition.

4. The composition of claim 1, wherein (a) is employed in an amount of from about 1 to about 10% by weight; (b) is employed in an amount of from about 0.5 to about 3% by weight; (c) is employed in an amount of from about 0.5 to about 3% by weight; (e) is employed in an amount of from about 1 to about 5% by weight; and (f) is employed in an amount of from about 1.0 to about 6.0% by weight (insert broadest range), all weights based on the total weight of the composition.

5. The composition of claim 1, further comprising Peumus boldus leaf extract employed in an amount of from about 0.1 to about 5% by weight, based on the total weight of the composition.

6. A composition intended for application onto human skin suffering from oxidative stress, the composition comprising: (1) a preservative system comprising: (a) from about 1 to about 5% by weight of a Lactobacillus ferment; (b) from about 1 to about 5% by weight, of a Lactobacillus and Cocos mucifera fruit extract; (c) up to about 0.5% by weight of salicylic acid; and (d) from about 0.1 to about 0.5% by weight of at least one salt of a weak acid; and (e) from about 1 to about 10% by weight of a petroleum-free 1,3-propanediol; (2) a mixture of at least: (f) a leaf extract of Aristotelia chilensis; (g) a leaf extract of Buddleja globosa; (h) a leaf extract of Ugni molinae; (i) optionally, a bark/seed extract of Entada phaseoloides; (j) optionally, a blend of Pfaffia paniculata, Ptychopetalum olacoides, and Lilium candidum extracts; and (k) at least one humectant, wherein (f)-(k) are employed in amounts sufficient to neutralize existing free radicals present in the skin, while proactively priming the skin in order to enhance its ability to defend itself against future free radical aggression; (3) an emulsifier; and (4) a dermatologically acceptable carrier, wherein the composition is natural, free of a skin sensitizing-effective amount of an essential oil, and has a pH ranging from about 4.5 to about 5.5.

7. The composition of claim 6, wherein (a) is employed in an amount of from about 2 to about 4% by weight; (b) is employed in an amount of from about 2 to about 4% by weight; (c) is employed in an amount of from about 0.1 to about 0.45% by weight; and (d) is employed in an amount of from about 0.2 to about 0.4% by weight; and (e) is employed in an amount of from about 2 to about 8% by weight, all weights based on the total weight of the composition.

8. The composition of claim 6, wherein (f) is employed in an amount of from about 1 to about 10% by weight; (g) is employed in an amount of from about 0.5 to about 3% by weight; (h) is employed in an amount of from about 0.5 to about 3% by weight; (i) is employed in an amount of from about 1 to about 5% by weight; (j) is employed in an amount of from about 1 to about 5% by weight; and (k) is employed in an amount of from about 1.0 to about 6.0% by weight, all weights based on the total weight of the composition, all weights based on the total weight of the composition.

9. The composition of claim 6, wherein (f) is employed in an amount of from about 2 to about 5% by weight; (g) is employed in an amount of from about 1 to about 2% by weight; (h) is employed in an amount of from about 1 to about 2% by weight; (i) is employed in an amount of from about 2 to about 3% by weight; (j) is employed in an amount of from about 2 to about 3% by weight; and (k) is employed in an amount of from about 1.5 to about 4.0% by weight, all weights based on the total weight of the composition, all weights based on the total weight of the composition.

10. The composition of claim 6, wherein (f) is employed in an amount of from about 1 to about 10% by weight; (g) is employed in an amount of from about 0.5 to about 3% by weight; (h) is employed in an amount of from about 0.5 to about 3% by weight; (i) is employed in an amount of from about 1 to about 5% by weight; and (k) is employed in an amount of from about 1.0 to about 8.0% by weight, all weights based on the total weight of the composition, all weights based on the total weight of the composition.

11. The composition of claim 6, wherein (f) is employed in an amount of from about 2 to about 5% by weight; (g) is employed in an amount of from about 1 to about 2% by weight; (h) is employed in an amount of from about 1 to about 2% by weight; (i) is employed in an amount of from about 2 to about 3% by weight; and (k) is employed in an amount of from about 2.0 to about 6.0% by weight, all weights based on the total weight of the composition, all weights based on the total weight of the composition.

12. The composition of claim 6, wherein (f) is employed in an amount of from about 1 to about 10% by weight; (g) is employed in an amount of from about 0.5 to about 3% by weight; (h) is employed in an amount of from about 0.5 to about 3% by weight; (j) is employed in an amount of from about 1 to about 5% by weight; and (k) is employed in an amount of from about 1.0 to about 8.0% by weight, all weights based on the total weight of the composition, all weights based on the total weight of the composition.

13. The composition of claim 10, wherein (f) is employed in an amount of from about 2 to about 5% by weight; (g) is employed in an amount of from about 1 to about 2% by weight; (h) is employed in an amount of from about 1 to about 2% by weight; (j) is employed in an amount of from about 2 to about 3% by weight; and (k) is employed in an amount of from about 2.0 to about 6.0% by weight, all weights based on the total weight of the composition, all weights based on the total weight of the composition.

14. The composition of claim 6, having a pH ranging from about 4.8 to about 5.3.

15. The composition of claim 6, further comprising Peumus boldus leaf extract employed in an amount of from about 0.1 to about 5% by weight, based on the total weight of the composition.

16. A method of simultaneously treating skin suffering from oxidative stress and proactively priming the skin in order to enhance its ability to defend itself against future free radical aggression by applying onto the skin a composition comprising: (1) a mixture of at least: (a) a leaf extract of Aristotelia chilensis; (b) a leaf extract of Buddleja globosa; (c) a leaf extract of Ugni molinae; (d) optionally, a bark/seed extract of Entada phaseoloides; (e) optionally, a blend of Pfaffia paniculata, Ptychopetalum olacoides, and Lilium candidum extracts; and (f) at least one humectant; (2) an emulsifier; and (3) a dermatologically acceptable carrier, wherein (a)-(f) are employed in amounts sufficient to neutralize existing free radicals present in the skin, while proactively priming the skin in order to enhance its ability to defend itself against future free radical aggression, thereby improving its health and appearance, and wherein the composition is natural and free of a skin sensitizing-effective amount of an essential oil.

17. The method of claim 16, wherein (a) is employed in an amount of from about 1 to about 10% by weight; (b) is employed in an amount of from about 0.5 to about 3% by weight; (c) is employed in an amount of from about 0.5 to about 3% by weight; (d) is employed in an amount of from about 1 to about 5% by weight; (e) is employed in an amount of from about 1 to about 5% by weight; and (f) is employed in an amount of from about 1.0 to about 8.0% by weight, all weights based on the total weight of the composition.

18. The method of claim 16, wherein (a) is employed in an amount of from about 1 to about 10% by weight; (b) is employed in an amount of from about 0.5 to about 3% by weight; (c) is employed in an amount of from about 0.5 to about 3% by weight; (d) is employed in an amount of from about 1 to about 5% by weight; and (f) is employed in an amount of from about 1.0 to about 6.0% by weight, all weights based on the total weight of the composition.

19. The method of claim 16, wherein (a) is employed in an amount of from about 1 to about 10% by weight; (b) is employed in an amount of from about 0.5 to about 3% by weight; (c) is employed in an amount of from about 0.5 to about 3% by weight; (e) is employed in an amount of from about 1 to about 5% by weight; and (f) is employed in an amount of from about 1.0 to about 8.0% by weight, all weights based on the total weight of the composition.

20. The method of claim 16, further comprising Peumus boldus leaf extract employed in an amount of from about 0.1 to about 5% by weight, based on the total weight of the composition.
